(19) United States
(12) Patent Application Publication
(10) Pub. No.: US 2025/0244319 A1
(43) Pub. Date: Jul. 31, 2025

(54) NANOWIRES USED AS ENZYME MIMICS

(71) Applicant: UNIVERSITY OF CENTRAL FLORIDA RESEARCH FOUNDATION, INC., Orlando, FL (US)

(72) Inventor: Xiaohu XIA, Orlando, FL (US)

(21) Appl. No.: 18/854,140

(22) PCT Filed: Apr. 4, 2023
(86) PCT No.: PCT/US2023/017412

§ 371 Date: Oct. 4, 2024

Related U.S. Application Data
(60) Provisional application No. 63/327,180, filed on Apr. 4, 2022.

(51) Int. Cl. G01N 33/543; B01J 23/46; B01J 31/16; B01J 31/22; B01J 35/45; B01J 35/58; B01J 37/16; B82Y 15/00; B82Y 40/00; G01N 33/574

(52) U.S. Cl. CPC G01N 33/54353; B01J 23/468; B01J 31/16; B01J 31/2226; B01J 35/45; B01J 35/58; B01J 37/16; G01N 33/57484; B01J 2231/005; B01J 2531/004; B01J 2531/827; B82Y 15/00; B82Y 40/00

(57) ABSTRACT

A class of nanowires that possess enzyme-like catalytic activities, which are termed as “enzyme mimics”. The morphologies of such enzyme mimics are nanoscale wires (i.e., nanowires) of various lengths, diameters, and degrees of branching and bending. The composition of such enzyme mimics may contain various elements, but at least one is a platinum-group metal [including platinum (Pt), iridium (Ir), rhodium (Rh), palladium (Pd), and/or ruthenium (Ru)]. The nanowires as enzyme mimics can efficiently catalyze multiple chemical reactions that are typically catalyzed by natural enzymes (such as peroxidase, catalase, oxidase, and superoxide dismutase). For example, the nanowires can act as peroxidase mimics and catalyze the oxidation of chromogenic substrates by oxidizing agents (e.g., hydrogen peroxide), yielding colored products. In addition, disclosed here include the applications of such enzyme mimics in biosensing systems, wherein the enzyme mimics arc conjugated to bioreceptors (e.g., antibodies) and are used as labels or reporters.

### GOVERNMENT SUPPORT CLAUSE

This invention was made with government support under Funding Award #1834874 awarded by the National Science Foundation. The government has certain rights in the invention.

### BACKGROUND

Enzymes (i.e., proteins that act as biological catalysts) are widely used in many biomedical applications such as biosensing, imaging, diagnostics, and therapy. Nevertheless, the inherent drawbacks of enzymes, including relatively low catalytic efficiency, relatively poor stability, and difficulties in storage/shipping, greatly limit the advancement of relevant biomedical technologies. In this invention, the inventor has developed a class of nanowires that possess enzyme-like catalytic activities, which are termed as “enzyme mimics” (or artificial enzymes). As advantages over natural enzymes, the present enzyme mimics are more catalytically efficient and stable owing to their optimized elemental compositions and morphologies. The enzyme mimics can be used as alternatives to natural enzymes for a broad range of applications in biomedicine.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1A-F are typical TEM images of different nanowires as enzyme mimics that are made of: (A) Ir, (B) Pt, (C) Rh, (D) Pd, (E) Ir—Pt alloy, and (F) Pt—Ni alloy. The 10 nm scale bar in (F) applies to all other images in (A-E).

FIG. 2 is a representative HAADF-STEM image taken from the Ir nanowires shown in FIG. 1A.

FIG. 3A-D are TEM images of Jr nanocrystals that were obtained using the standard synthetic procedure for Ir nanowires shown in FIG. 1A, except that the concentration of Na₃CA solution was changed from 4.5 mg/mL to (A) 1.7 mg/mL; (B) 6 mg/mL, (C) 8 mg/mL; and (D) 11 mg/mL. The 10 nm scale bar in (D) applies to all other images in (A-C).

FIG. 4A-B are TEM images of Pd nanowires that were obtained using the standard synthetic procedure of Pd nanowires shown in FIG. 1D, except that the concentration of Na₃CA solution was changed from 3.5 mg/mL to (A) 2 mg/mL; and (B) 5 mg/mL; The average diameters of the nanowires in (A) and (B) were roughly 5.5 and 9 nm, respectively.

FIG. 5A-D are high resolution HAADF-STEM images taken from different nanowires (samples shown in FIG. 1): (A) Ir nanowires; (B) Pt nanowires; and (C) Ir—Pt alloyed nanowires. (D) Magnified HAADF-STEM image of the region marked by a dashed yellow box in (B). The white arrows in (A-D) indicate twin defects in nanowires.

FIG. 6 are XRD patterns recorded from nanowires (samples shown in FIG. 1) of different metals as labeled in the spectra. The nanowires were loaded on carbon support for XRD analyses.

FIG. 7A-H are XPS spectra recorded from different nanowires (samples shown in FIG. 1): (A, B) Ir nanowires; (C, D) Pt nanowires; (E, F) Ir—Pt alloyed nanowires; and (G, H) Pt—Ni alloyed nanowires.

FIG. 8A-D are EDS analyses of a single Ir—Pt alloyed nanowire: (A) HAADF-STEM image of a single Ir—Pt nanowire; (B) EDS mapping image of the same nanowire shown in (A); (C) and (D) line-scan EDS spectra of elemental Ir and Pt that were recorded from the nanowire shown in (A) along the directions indicated by arrows #1 and #2, respectively.

FIG. 9 is FTIR spectra recorded from aqueous solution of pure sodium citrate and aqueous suspension of citrate-capped Ir nanowires shown in FIG. 1A. Peak assignments are labeled on the spectra.

FIG. 10A-B shows the peroxidase-like catalytic activities of the Ir nanowires shown in FIG. 1A. (A) Photographs taken from various peroxidase substrate solutions with and without nanowires. The nanowires could catalyze the oxidations of TMB, ABTS, DAB, and OPD by H2O2, producing blue-, blue/green-, brown-, and orange-colors products, respectively; (B) UV-vis spectrum taken from the sample #5 in (A).

FIG. 11 is a plot of initial reaction velocity (v) against TMB concentration. The plot was derived from the kinetic assay of Ir nanowires- (sample in FIG. 1A) catalyzed oxidation of TMB by H2O2.

FIG. 12 shows the catalase-like catalytic activities of various nanowires shown in FIG. 1A. Photographs taken from 1 M H₂O₂ solutions (pH 10.0) in the absence (#1) and presence (#2-7) of nanowires. The solutions were hold in centrifuge cubes. The nanowires could catalyze the conversion of hydrogen peroxide to water and oxygen, producing oxygen bubbles in the tubes.

FIG. 13A-B are schematics showing the principles of (A) nanowires-based ELISA, and (B) HRP-based ELISA.

FIG. 14 are calibration curves of Ir nanowires- and HRP-based ELISAs of CEA standards. The same antibodies were used in both ELISAs.

FIG. 15A-D are calibration curves of different nanowires-based ELISAs of CEA standards as indicated in the plots. The same antibodies were used in all the ELISAs. Linear detection ranges in the plots were indicated by straight lines.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “nanowire” generally refers to any elongated nanocrystal or shape that includes at least one cross sectional dimension that is less than 500 nm, and typically, less than 100 nm. In specific examples, nanowires have an aspect ratio (i.e., length to diameter ratio) greater than 3 (>3)

The term “platinum-group metal” refers to the six noble, precious metallic elements clustered together in the periodic table. These elements are all transition metals in the d-block (groups 8, 9, and 10, periods 5 and 6). The six platinum-group metals are ruthenium, rhodium, palladium, osmium, iridium, and platinum. They have similar physical and chemical properties, and tend to occur together in the same mineral deposits. However, they can be further subdivided into the iridium-group platinum-group elements (IPGEs: Os, Ir, Ru) and the palladium-group platinum-group elements (PPGEs: Rh, Pt, Pd) based on their behaviour in geological systems.

The term “bioreceptor” refers to a biological element (e.g., enzyme, antibody, aptamer, nucleic acid, biotin, avidin etc.) which selectively interacts with or binds to (senses) to another molecule such as an analyte (e.g., enzyme substrate, complementary nucleic acid, antigen). A bioreceptor may interact with a specific analyte of interest to produce an effect measurable by a transducer. High selectivity for the analyte among a matrix of other chemical or biological components is a key requirement of a bioreceptor. Common types of bioreceptor interactions involve: antibody/antigen, enzymes/ligands, nucleic acids/DNA, biotin/avidin, cellular structures/cells, or biomimetic materials. The bioreceptor may be conjugated to a nanowire. Detection of an analyte may involve direct or indirect sensing by a nanowire comprising a bioreceptor. For example, indirect sensing would include use in an assay involving other detection molecule(s) that directly bind to the analyte and the bioreceptor on the nanowire binds to the detection molecule.

The terms “enzyme-like catalytic activity” as used herein refers to an artificial enzyme (e.g. nanowire) that mimics the activity of an enzyme.

The term “biomolecule” as used herein refers to antibodies, proteins, carbohydrates peptides, lipids, biotin/avidin, nucleic acids, glycoproteins, lipoproteins and glycolipids.

The term “chemical ligand” as used herein refers to a molecule on a nanowire that is a remnant of a capping agent or stabilizer that is used in the synthesis of nanowires. For example, citrate is a remnant of use of sodium citrate when combined with a metal precursor during synthesis.

### DETAILED DESCRIPTION

Described herein is a class of nanowires that possess enzyme-like catalytic activities, which will be termed as “enzyme mimics” in the following. The morphologies of such enzyme mimics are nanoscale wires (i.e., nanowires). The nanowires may be wavy or straight and may have various degrees of branching or have no branches. The composition of such enzyme mimics may contain various elements, but at least one is a platinum-group metal [including platinum (Pt), iridium (Ir), rhodium (Rh), palladium (Pd), and/or ruthenium (Ru)]. As examples, the enzyme mimics may be composed of platinum-group metal(s) only, a mix of platinum-group metal(s) and other metals (e.g., in the forms of alloys and/or core-shell structures), a mix of platinum-group metal(s) and non-metal elements (e.g., in the form of compounds such as metal oxides), and/or a combination of platinum-group metal(s) and other nanostructures/molecules (e.g., in the form of composites). The nanowires as enzyme mimics can efficiently catalyze multiple chemical reactions that are typically catalyzed by natural enzymes. For instance, the nanowires can act as peroxidase mimics and catalyze the oxidation of chromogenic substrates by oxidizing agents (e.g., hydrogen peroxide), yielding colored products.¹ The enzyme mimics can be used as alternatives to natural enzymes for a broad range of biomedical applications such as biosensing and diagnostics.

### 1. Synthesis

The nanowires are prepared using a solution-phase synthesis, where metal precursors are reduced by sodium borohydride (NaBH₄) as a reductant in the presence of sodium citrate (Na₃CA) as a stabilizer.

In a specific embodiment, the nanowire is comprised of iridium (Ir). A standard synthetic procedure is described in the following: To a 100 mL-flask, 18 mL deionized water, 2 mL aqueous solution of Na₃IrCl₆·xH₂O (Ir precursor, 4.77 mg/mL), and 3 mL aqueous solution of Na₃CA (4.5 mg/mL) are added. Under magnetic stirring, 12 L of 1 M NaOH solution is added to the flask. The flask is then placed in a 130° C. oil bath under magnetic stirring and is degassed with a flow of nitrogen (N₂) gas for ˜7 minutes. Then under N₂ blanket, 1 mL aqueous solution of NaBH₄ (3.8 mg/mL, prepared with cold water) is quickly injected to the flask. Immediately after the injection of NaBH₄ solution, the flask is taken out from the oil bath and placed on a stirrer plate. After stirring at room temperature for 45 minutes, the final products (i.e., Ir nanowires) are collected by centrifugation and washed once with deionized water. Finally, the products are re-dispersed in deionized water for future use.

Nanowires of other metals are synthesized using the same synthetic procedure described for Ir nanowire synthesis, except that: i) the Na₃IrCl₆·xH₂O solution is replaced with solutions of other metal precursors at the same molar concentration; and ii) the concentration of Na₃CA solution may be varied.

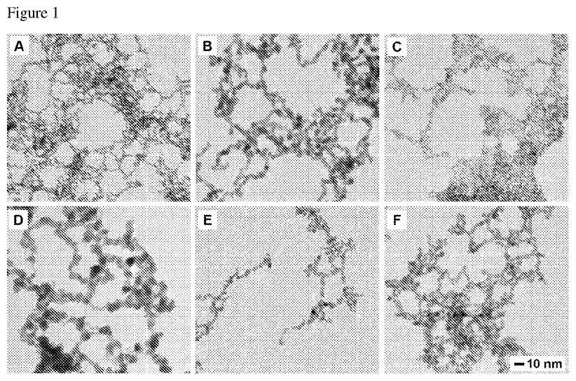

In certain embodiments, Pt, Rh, Pd, Ir—Pt alloyed, and Pt-nickel (Ni) alloyed nanowires (samples in FIG. 1) are synthesized when Na₂PtCl₆·6H₂O, Na₃RhCl₆, Na₂PdCl₄, a mix of Na₃IrCl₆·xH₂O and Na₂PtCl₆·6H₂O, and a mix of Na₂PtCl₆·6H₂O and NiCl₂·6H₂O are used as precursors, respectively; and the concentrations of Na₃CA solution can be set to be 3.5, 2.0, 3.5, 2.0, and 3.5 mg/mL, respectively.

Alternative metal precursors may be used for the synthesis of the same nanowires. For multi-metallic nanowires (including bimetallic nanowires), the atomic ratio of metals in the nanowires can be controlled by adjusting the amounts of different metal precursors.

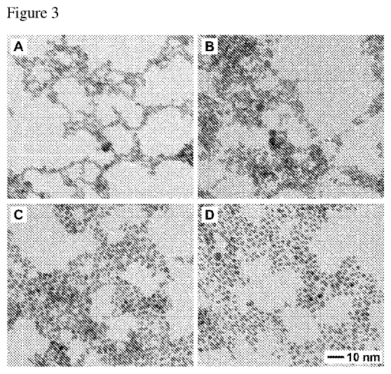

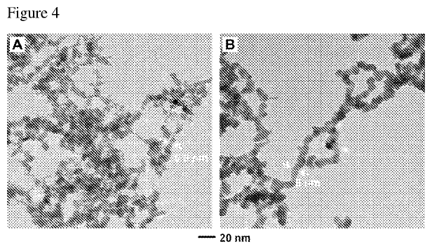

It should be emphasized that the overall length, degree of branching, and diameter of nanowires can be controlled by adjusting the concentration of Na₃CA or other reagents in the synthetic reaction system (see, FIGS. 3 and 4).

### 2. Characterizations

### i) Morphologies

Morphologies (including size and shape) of the nanowires are characterized by transmission electron microscopy (TEM) and high-angle annular dark field scanning TEM (HAADF-STEM). FIG. 1 shows TEM images of representative samples of various nanowires. It can be seen that the samples display an overall wire-like morphology. The nanowires are wavy and exhibit variable degrees of branching and diameter. The diameters of the nanowires in FIG. 1 are in the range of ˜2-10 nm. For instance, the average diameters of Ir nanowires (FIG. 1A) and Pt nanowires (FIG. 1B) are approximately 2 nm and 4 nm, respectively.

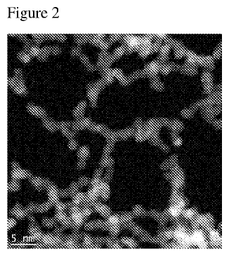

The morphology of nanowires can also be revealed by HAADF-STEM imaging. In a specific embodiment, the Ir nanowire sample shown in FIG. 1A is characterized by HAADF-STEM and the image is shown in FIG. 2.

The overall length and degree of branching of nanowires could be controlled by adjusting the concentration of Na₃CA solution (or other reagents) in the synthetic system. Taking Ir nanowires as an example, when the concentration of Na₃CA solution in a standard synthesis is increased, the overall length and degree of branching of Ir nanowires are decreased (see FIG. 3).

The diameter of nanowires can be tuned by varying the concentration of Na₃CA solution (or other reagents) in the synthetic system. With Pd nanowires, the average diameters of Pd nanowires can be changed from ˜8 nm to ˜5.5 nm and ˜9 nm (see FIG. 4), respectively, when the concentration of Na₃CA solution in a synthesis is changed from 3.5 mg/mL to 2 mg/mL and 5 mg/mL.

### ii) Crystal Structures

Crystal structures of the nanowires are characterized by high-resolution HAADF-STEM and X-ray diffraction (XRD). The results show that the nanowires take a face-centered cubic (fcc) structure. Twin defects can be observed in the nanowires.

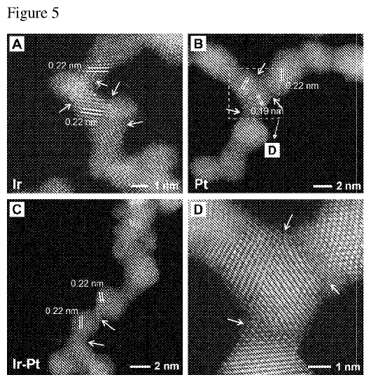

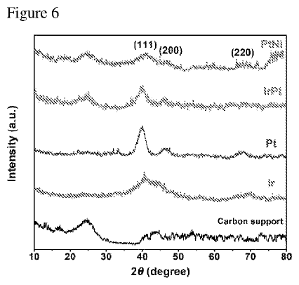

High-resolution HAADF-STEM images of the Ir, Pt, and Ir—Pt alloyed nanowires are shown in FIG. 5. XRD patterns recorded from different nanowires are shown in FIG. 6. The broadening of peaks in XRD patterns can be ascribed to the relatively small cross sections of the nanowires.

iii) Elemental Compositions

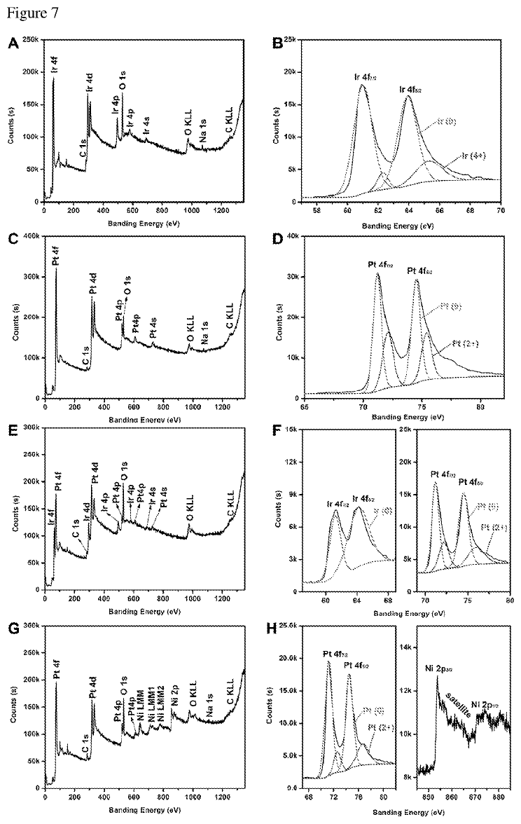

Elemental compositions of the nanowires are confirmed by XRD (FIG. 6) and X-ray photoelectron spectroscopy (XPS). Since the nanowires are relatively thin, the X-ray in XPS spectrometer is expected penetrate to the cores of individual nanowires. As such, in this case, XPS data can be used to analyze the elemental compositions of nanowires. As shown by the XPS spectra in FIG. 7, the nanowires are primarily made of zero-valent metals that originate from the reduction of metal precursors during synthesis.

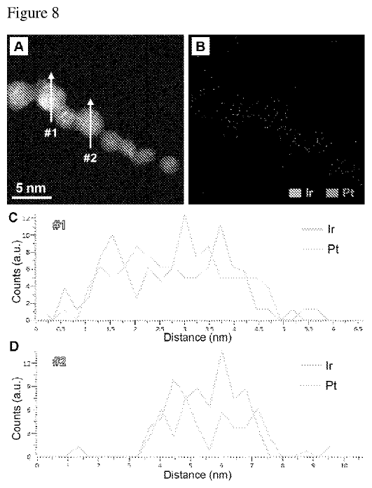

For multi-metallic nanowires, elemental composition and distributions of elements in individual nanowires can be analyzed by energy-dispersive X-ray spectroscopy (EDS). For Ir—Pt alloyed nanowires (sample in FIG. 1E), FIG. 8 shows EDS mapping images and line-scan spectra of a single nanowire. Ir and Pt elements are confirmed to co-exit in the nanowires in the form of alloy.

### iv) Surface Ligands

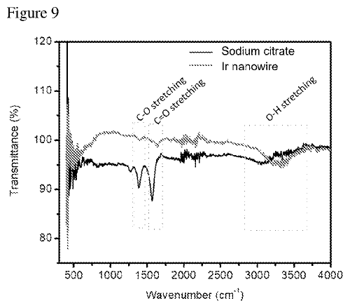

The surfaces of the nanowires are absorbed or capped with a remnant of stabilizer, such as citrate, wherein the stabilizer, such as sodium citrate, is initially introduced to the synthetic solution and acts as a colloidal stabilizer. Taking Ir nanowires (sample in FIG. 1A), the presence of citrate on nanowire surfaces is confirmed by Fourier Transform Infrared Spectroscopy (FTIR) analysis (see FIG. 9).

It should be emphasized that the citrate molecules on nanowire surfaces can be replaced by other chemical ligands (e.g., polyvinylpyrrolidone, cetyltrimethylammonium chloride/bromide, and polyethylene glycol) through various methods (e.g., ligand exchange). In addition, by introducing different chemical ligands as stabilizers or capping agents to the synthetic reaction solution, the nanowires can be capped by various types of ligands on the surfaces.²

### 3. Enzyme-Like Activities

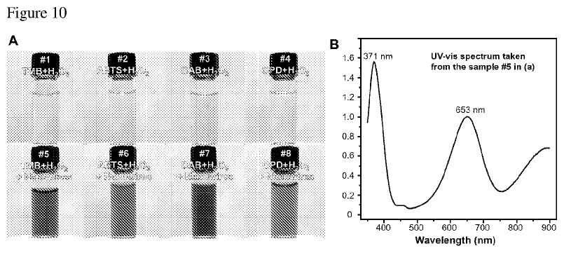

In certain embodiments, the nanowires possess enzymatic mimetic activity, such as peroxidase-like catalytic activities.¹ Specifically, in certain embodiments, the nanowires can effectively catalyze the oxidation of peroxidase substrates [e.g., 3,3′,5,5′-tetramethylbenzidine, TMB; 3,3′-diaminobenzidine, DAB; o-phenylenediamine, OPD; and 2,2′-Azino-bis(3-ethylbenzothiazoline-6-sulfonic acid) diammonium salt, ABTS] by oxidizing agents (e.g., hydrogen peroxide), yielding colored products. See more details in FIGS. 9 and 10 and associated discussions below.

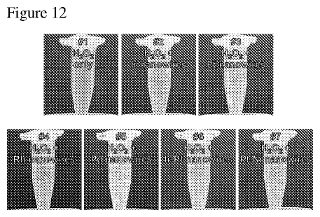

In addition to peroxidase, the nanowires possess similar catalytic activities of other enzymes such as catalase, oxidase, and superoxide dismutase. In certain embodiments, the nanowires of different metals (samples shown in FIG. 1) can effectively catalyze the conversion of hydrogen peroxide to water and oxygen (O₂) in aqueous solution, generating oxygen bubbles (see FIG. 12). These results suggest that the nanowires possess catalase-like catalytic activities.

### 4. Conjugation with Biomolecules

The nanowires can be conjugated with various biomolecules (e.g., antibodies, proteins, peptides, biotin/avidin, and nucleic acids) through covalent methods (e.g., metal-thiolate bonding-mediated couplings) and/or non-covalent methods (e.g., electrostatic and hydrophobic interactions).^{3,4} In a specific embodiment directed to conjugation of antibodies with a non-covalent method, a typical procedure is described in the following:

An aqueous suspension of nanowires is adjusted, if needed, to pH 8-10 with carbonate-bicarbonate buffer or NaOH. Then, certain amount of antibody is added to the nanowire suspension. After 1-hour incubation at room temperature, a quantity of 5% bovine serum albumin (BSA) is added to the suspension. After 2-hour incubation, the nanowire-antibody conjugates are collected by centrifugation and washed twice by phosphate-buffered saline (PBS, pH 7.4) buffer. Finally, the conjugates are re-dispersed in PBS containing 1% BSA and 0.02% NaN₃ for future use. The loading amount of antibodies on nanowires can be adjusted by varying the ratio of nanowires and antibodies used in the conjugation.

### 5. Applications in Biosensing

In certain embodiments, nanowires can be conjugated with a bioreceptor. When conjugated with bioreceptors (e.g., antibodies, nucleic acids, and aptamers), the nanowires can be used as labels or reporters for various biosensing and diagnostic applications such as enzyme-linked immunosorbent assay (ELISA), western blotting, lateral flow assay, and immunohistochemistry.¹ The nanowires-based assays can be used for sensing various types of analytes, such as biomarkers, chemicals, drugs, virus, and pathogens, as long as appropriate bioreceptors are available.

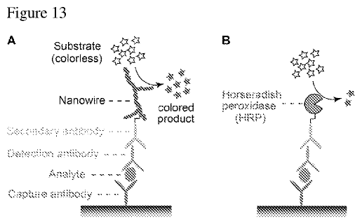

Taking ELISA platform as a model biosensing system, FIG. 13 shows a typical detection principle of the nanowires-based ELISA. Herein, the nanowires act as peroxidase mimics that can catalyze the oxidation of peroxidase substrates (e.g., 3,3′,5,5′-tetramethylbenzidine, TMB) by H₂O₂ to form colored products. The principle of such a nanowires-based ELISA is essentially the same as conventional horseradish peroxidase (HRP)-based ELISA, except for the substitution of HRPs with nanowires as peroxidase mimics. It should be mentioned that, in this design, nanowires are conjugated to a secondary antibody (e.g., goat anti-mouse IgG) in order to make the platform generic. However, depending on the types of target analyte and needs, one can use the nanowires to design various ELISAs of different formats (e.g., direct, indirect, sandwich, and competitive ELISAs). In addition, one can conjugate nanowires to other functional molecules (e.g., biotin, avidin, and polymers) to achieve different designs.

### EXAMPLES

### Example 1. Ir Nanowires-Catalyzed Reactions

FIG. 10 and its caption show the results from Ir nanowires-catalyzed reactions. The same results were obtained when nanowires of other metals (samples shown in FIG. 1B-F) were used as catalysts. These results suggest that the nanowires possess peroxidase-like catalytic activities.

Taking TMB as a model substrate, the catalytic reaction yielded a blue-colored product (i.e., oxidized TMB) with a maximum absorbance at 653 nm (see FIG. 10B). Like natural peroxidases, the catalytic activity of nanowires as peroxidase mimics is dependent on pH, temperature, and TMB and H₂O₂ concentrations. A typical catalytic reaction was carried out in a 1.0 mL of 0.2 M sodium acetate/acetic acid (NaOAc/HOAc) buffer solution pH 4.0, containing certain amount of nanowires as the catalysts and 2 M H₂O₂ and 0.8 mM TMB as the substrates.

The peroxidase-like catalytic efficiency of nanowires can be determined by the steady-state kinetic assays, where oxidation of TMB by H₂O₂ is used as a model reaction.⁵ Using Ir nanowires (sample shown in FIG. 1A) as an example, typical procedures of the kinetic assays are described in the following:

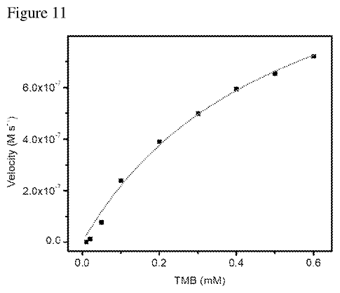

All the assays were conducted in 1.0-mL cuvettes (path length, L=1.0 cm) and 0.2 M NaOAc/HOAc buffer, pH 4.0.5 In a typical assay, Ir nanowires (final concentration in reaction solution=1.0 ng/mL. Note “ng” refers to nanogram or 10⁻⁹ gram) were mixed with 2.0 M H₂O₂ and TMB of various concentrations in the buffer solution. The absorbance of reaction solution at λₘₐₓ=653 nm was immediately recorded by a UV-vis spectrophotometer with an interval of 2 seconds for 1 minute. The “Absorbance versus Time” curve was obtained, which was then used to derive the initial reaction velocity (ν) through the equation: ν=Slopeᵢₙᵢₜᵢₐₗ/(ε_{TMS-653 nm}×L). Herein, Slopeᵢₙᵢₜᵢₐₗ is the first derivation from the initial point on the measured curve, ε_{TMB-653 nm} is the molar extinction coefficient of oxidized TMB at 653 nm (3.9×10⁴ M⁻¹·cm⁻¹) and L is the path length (1.0 cm). Then, the plot of initial reaction velocity (ν) against TMB concentration (see FIG. 11) was obtained and fitted by the nonlinear regression of the Michaelis-Menten equation ν=Vₘₐₓ×[S]/(Kₘ+[S]). Wherein, Vₘₐₓ represents the maximal reaction velocity, [S] is the concentration of TMB, and Kₘ is the Michaelis constant or the concentration of TMB at which the reaction velocity is at half-maximum.

From the plot shown in FIG. 11, Vₘₐₓ and Kₘ for Ir nanowires were determined to be 1.346×10⁻⁶ M s⁻¹ and 0.515 mM, respectively, by using Origin software. Based on the values of Vₘₐₓ and concentration of Ir nanowires in the reaction solution (1.0 ng/mL), the catalytic efficiency (defined as the maximum number of colored products generated per second per unit mass of catalyst) of Ir nanowires was estimated to be 8.1×10¹⁴ s⁻¹ ng⁻¹. The catalytic efficiencies of other types of nanowires could be estimated using the same method.

### Example 2. Nanowire-Based ELISA

Using carcinoembryonic antigen (CEA, a cancer biomarker) as a model analyte, a typical procedure of nanowires-based ELISA is described in the following: A 96-well microtiter plates was coated with rabbit anti-CEA polyclonal antibody (corresponding to the “capture antibody” in FIG. 13). The plate was washed with washing buffer (10 mM PBS pH 7.4 containing 0.05% Tween 20, PBST). Then, the plate was blocked by blocking buffer (PBST containing 3% BSA) for 2 hours at room temperature, followed by washing. 100 μL CEA standards or samples were added to the wells of plate, followed by shaking at room temperature for 2 h. After washing, mouse anti-CEA monoclonal antibody (corresponding to the “detection antibody” in FIG. 13) was added, followed by shaking at room temperature for 1 h and three times washing. To the wells of plate, nanowire-goat anti-mouse IgG conjugates at a certain dilution factor was added. The plate was incubated at room temperature for 1 h. After washing, 100 μL freshly prepared substrate solution (0.2 M NaOAc/HOAc buffer solution pH 4.0, containing 0.8 mM TMB and 2.0 M H₂O₂) was added to each well. After 5-30 min, 50 μL of 2.0 M H₂SO₄ as stopping solution was added to each well. Then the value of absorbance at 450 nm of each well was measured by a microplate reader. Optimally, the background absorbance may be subtracted from all data points prior to plotting the absorbance against analyte concentration.

For comparison, HRP-based ELISA using the same set of antibodies was also performed. The assay procedure and materials of HRP-based ELISA were kept the same as the nanowires-based ELISA, except for the substitutions of nanowire-goat anti-mouse IgG conjugates with HRP-goat anti-mouse IgG conjugates and the change of substrate solution to citric acid/Na₂HPO₄ buffer pH 5.0, containing 0.8 mM TMB and 5 mM H₂O₂.

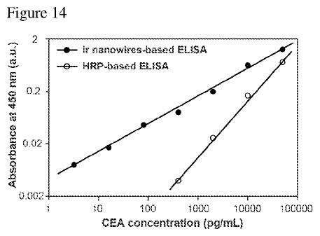

FIG. 14 shows the calibration curves of Ir nanowires-based ELISA and HRP-based ELISA, where CEA standards in a mix of dilution buffer (PBST containing 1% BSA) and negative human serum (purchased from Sigma Aldrich) at a volume ratio of 1:1 were detected. From the calibration curves shown in FIG. 14, the limits of detection (LODs) of Ir nanowires- and HRP-based ELISAs were determined to be approximately 3.2 pg/mL and 400 pg/mL, respectively. Note, “pg” refers to picogram or 10⁻¹² gram. The Ir nanowires-based ELISA displayed a good linear detection range of 3.2-5000 pg/mL.

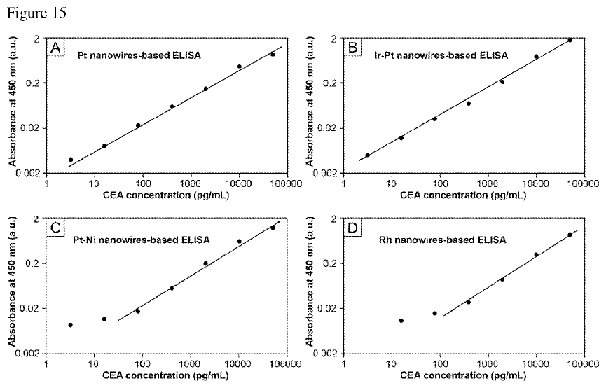

Pt nanowires-, Ir—Pt nanowires-, Pt—Ni nanowires-, and Rh nanowires-based ELISAs of CEA standards were performed in separate experiments. Their calibration curves are shown in FIG. 15.

### REFERENCES

1. Wei, Z.; Xi, Z.; Vlasov, S.; Ayala, J.; Xia, X. Nanocrystals of platinum-group metals as peroxidase mimics for in vitro diagnostics. Chemical Communications, 2020, 56, 14962-14975.
2. Yang, T.; Shi, Y.; Janssen, A.; Xia, Y. Surface capping agents and their roles in shape-controlled synthesis of colloidal metal nanocrystals. Angewandte Chemie International Edition, 2020, 59, 15378-15401.
3. Sperling, R. A.; Parak, W. J. Surface modification, functionalization and bioconjugation of colloidal inorganic nanoparticles. Philosophical Transactions: Mathematical, Physical and Engineering Sciences, 2010, 368, 1333-1383.
4. Jazayeri, M. H.; Amani, H.; Pourfatollah, A. A.; Pazoki-Toroudi, H.; Sedighimoghaddam, B. Various methods of gold nanoparticles (GNPs) conjugation to antibodies. Sensing and Bio-Sensing Research, 2016, 9, 17-22.
5. Xia, X.; Zhang, J.; Lu, N.; Kim, M.; Ghale, K.; Xu, Y.; McKenzie, E.; Liu, J.; Ye, H. Pd—Ir core-shell nanocubes: a type of highly efficient and versatile peroxidase mimic. ACS Nano, 2015, 9, 9994-10004.

What is claimed is:

1. A nanowire with enzyme-like catalytic activities comprised of at least one platinum-group metal, and a chemical ligand, wherein the chemical ligand is located on the surface of the nanowire.

2. The nanowire of claim 1, wherein the platinum-group metal is selected from the group consisting of platinum (Pt), iridium (Ir), rhodium (Rh), palladium (Pd), or ruthenium (Ru).

3. The nanowire of claim 1, wherein the chemical ligand comprises citrate, polyvinylpyrrolidone, cetyltrimethylammonium chloride/bromide, or polyethylene glycol.

4. The nanowire of claim 1, further comprising one or more non-platinum-group element.

5. The nanowire of claim 1, wherein the nanowire comprises a diameter of less than 100 nm.

6. The nanowire of claim 5, wherein the nanowire comprises a diameter of 2 to 10 nm.

7. The nanowire of claim 1, wherein the nanowire comprises an aspect ratio of greater than 3.

8. The nanowire of claim 1, wherein the enzyme-like activities comprise peroxidase activity, catalase activity, oxidase activity, or superoxide dismutase activity.

9. The nanowire of claim 1, wherein the nanowire is conjugated with a biomolecule.

10. The nanowire of claim 10, wherein the biomolecule comprises an antibody, a protein, a peptide, biotin/avidin, a nucleic acid, or an aptamer.

11. A method for synthesizing a nanowire with catalytic activities comprising combining one or more platinum-group metal precursors and a stabilizer into a solution, stirring said solution under a first condition comprising a first temperature, the first temperature optionally comprising about 130° C., degassing said solution with a flow of nitrogen gas, injecting a reductant into said solution, stirring said solution under a second condition comprising a second temperature, the second temperature optionally comprising about 20° C., and finally collecting and washing the nanowire from said solution.

12. The method of claim 11, wherein the platinum-group metal is selected from the group consisting of platinum (Pt), iridium (Ir), rhodium (Rh), palladium (Pd), or ruthenium (Ru).

13. The method of claim 11, wherein the stabilizer comprises sodium citrate (Na₃CA).

14. The method of claim 13, wherein the Na₃CA has a concentration of 1.7 mg/mL to 11 mg/mL.

15. The method of claim 11, wherein the reductant comprises sodium borohydride (NaBH₄).

16. The method of claim 15, wherein the NaBH₄ has a concentration of 3.8 mg/mL.

17. A biosensing system for an assay comprising the nanowire of claim 9, wherein the biomolecules is a bioreceptor.

18. The system of claim 17, wherein the bioreceptor comprises an antibody, a nucleic acid, an avidin, and an aptamer.

19. The system of claim 17, wherein the assay comprises an ELISA, a western blot, a lateral flow assay, or an immunohistochemistry assay.

20. The system of claim 17, wherein the system senses a biomarker, a chemical, a drug, a virus, or a pathogen.

21. A nanowire with enzyme-like catalytic activities comprised of one or more platinum-group metal, one or more non-platinum-group elements, and a chemical ligand, wherein the chemical ligand is located on the surface of the nanowire.

22. A method of detecting an analyte in a sample, the method comprising exposing the sample to a nanowire of claim 9, wherein the biomolecule is a bioreceptor that senses the analyte or another molecule bound to the analyte.

23. The method of claim 22, wherein the exposing is a step of an ELISA, a western blot, a lateral flow assay, or an immunohistochemistry assay.
